(19) United States
(12) Patent Application Publication
(10) Pub. No.: US 2015/0225686 A1
(43) Pub. Date: Aug. 13, 2015

(54) METHOD FOR DETACHING CELLS FROM ADHESION SURFACE AND CELL DETACHMENT SYSTEM

(71) Applicant: TOKYO ELECTRON LIMITED, Minato-ku (JP)

(72) Inventors: Yasuhiro OSHIMA, Stevenage (GB); Shinichi GOMI, Minato-ku (JP); Kunitada HATABAYASHI, Minato-ku (JP); Kentarou TOMITA, Minato-ku (JP); Tomoaki KURAKAZU, Stevenage (GB)

(73) Assignee: TOKYO ELECTRON LIMITED, Minato-ku (JP)

(21) Appl. No.: 14/620,306

(22) Filed: Feb. 12, 2015

(30) Foreign Application Priority Data
Feb. 13, 2014 (JP) 2014-025746

(51) Int. Cl. C12M 1/00; C12N 5/074; C12N 13/00

(52) U.S. Cl. CPC C12M 31/02; C12N 13/00; C12M 23/22; C12N 5/0696

(57) ABSTRACT

A method for detaching cells from a cell culture surface includes irradiating visible light in an irradiation amount of 4 J or greater per 1 mm² to a cell adhesion surface to which cells are adhering on a substrate such that the cells are detached from the cell adhesion surface on the substrate.

### CROSS-REFERENCE TO RELATED APPLICATIONS

The present application is based upon and claims the benefit of priority to Japanese Patent Application No. 2014-025746, filed Feb. 13, 2014, the entire contents of which are incorporated herein by reference.

### BACKGROUND OF THE INVENTION

### 1. Field of the Invention

The present invention relates to a method for detaching cells from an adhesion surface, and to a cell detachment system.

2. Description of Background Art

Japanese Patent Laid-open Publication No. 2012-125218 describes a method that when a photolytic coating on a culture vessel surface is subjected to photoirradiation, the coating decomposes, cells are damaged by acid that is produced and the cells thereby detach from an adhesion surface. In addition, in Japanese Patent Laid-open Publication No. 2012-80844, a photoresponsive gel dissolves when the gel is subjected to photoirradiation, thereby causing cells adhered to a surface to detach. The entire contents of these publications are incorporated herein by reference.

### SUMMARY OF THE INVENTION

According to one aspect of the present invention, a method for detaching cells from a cell culture surface includes irradiating visible light in an irradiation amount of 4 J or greater per 1 mm² to a cell adhesion surface to which cells are adhering on a substrate such that the cells are detached from the cell adhesion surface on the substrate.

According to another aspect of the present invention, a cell detachment system includes a cell culture vessel having a cell adhesion surface to which cells adhere, and an irradiation device which includes a light source for cell detachment and irradiates visible light to the cell adhesion surface on the cell culture vessel. The cell culture vessel includes a vessel wall transparent to the visible light irradiated from the irradiation device, and the light source of the irradiation device is positioned outside the cell culture vessel and irradiates the visible light which transmits through the vessel wall to the cell adhesion surface of the cell culture vessel.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 shows photographs illustrating cell detachment due to irradiation with light having a wavelength of 420 nm;

FIG. 2 shows photographs illustrating cell detachment due to irradiation with light having wavelengths of 405 nm and 450 nm;

FIG. 3 is a schematic diagram illustrating a cell detachment system according to a first embodiment of the present invention;

FIG. 4 is a cross-sectional view along a line A-A in FIG. 3; and

FIG. 5 is a cross-sectional view illustrating an exemplary modification of the cell detachment system according to the first embodiment of the present invention.

### DETAILED DESCRIPTION OF THE EMBODIMENTS

The embodiments will now be described with reference to the accompanying drawings, wherein like reference numerals designate corresponding or identical elements throughout the various drawings.

The word “cell” as used in the present invention is not particularly limited so long as the cell is capable of being used in an adhesive culture. The cell can be a mammalian cell such as a primate cell or rodent cell, and is preferably a human, monkey, mouse, rat, marmot, hamster, rabbit, cat, dog, sheep, pig, cow, or goat cell, or a cell derived from the same. The cells used in the present invention can be used without particular limitation, regardless of the organ or tissue from which the cells are derived. Preferable examples can include somatic cells such as nerve cells, muscle cells, heart cells, lung cells, kidney cells, liver cells, epithelial cells, endothelial cells, or fibroblasts; or stem cells, germ cells, or pluripotent stem cells of the same. Preferably, pluripotent stem cells can be used. Examples of the pluripotent stem cells used in the present invention include embryonic stem cells (ES cells), induced pluripotent stem cells (iPS cells or artificial pluripotent stem cells), Muse cells (MUltilineage-differentiating Stress Enduring cells), STAP cells (Stimulus-Triggered Acquisition of Pluripotency cells), embryonal carcinoma cells (EC cells), or embryonic germ stem cells (EG cells). ES cells or iPS cells are preferred. The pluripotent stem cells used in the present invention are, more preferably, mammalian pluripotent stem cells of a primate or rodent, for example; still more preferably, mammalian ES cells or iPS cells of a primate or rodent, for example; and most preferably human ES cells or iPS cells. In the present invention, one of cells which proliferate in a single layer on an adhesion surface and cells which proliferate in multiple layers may be used.

The “adhesion surface” used in the present invention refers to a surface of a culture vessel to which cells are adhered, and may be an adhesion surface of an open system culture vessel or a closed system culture vessel. Although the “adhesion surface” used in the present invention is not particularly limited, the adhesion surface is a surface suitable for cell culture, and preferably is a surface of a light-transmissive substrate. By using a light-transmissive substrate as a vessel wall, when light is irradiated from an exterior of the culture vessel, detachment of cells from the adhesion surface is enabled due to the light transmitting through the vessel wall. The light-transmissive substrate should be transparent at least to the light to be irradiated. The substrate forming the cell adhesion surface may be an elastic substrate such as a film, or may be a hard, non-elastic substrate. The substrate can, for example, be made of plastic, glass, or quartz.

The “adhesion surface” used in the present invention is not necessarily subjected to a coating process but, from a standpoint of improving adhesion efficiency of cells, coating with a cell adhesion coating is preferred. Examples of the cell adhesion coating are not particularly limited, and can include polymer coatings or extracellular matrix (ECM) coatings such as gelatin, collagen, laminin, entactin, hyaluronic acid, Matrigel (registered trademark, manufactured by BD & Co.), CELLstart (registered trademark, manufactured by Invitrogen), Pronectin (registered trademark, manufactured by Wako Pure Chemicals Co.), adhesive oligopeptide, polylactide, polyglycolic acid, polylysine, or polyornithine. In particular, a surface coating using gelatin, collagen, Matrigel (registered trademark, manufactured by BD & Co.), CELLstart (registered trademark, manufactured by Invitrogen), or Pronectin (registered trademark, manufactured by Wako Pure Chemicals Co.) is preferably used as an adhesion coating for pluripotent stem cells. The cell adhesion coating can be applied using a method detailed in a manufacturing operator's operation manual, or by some other commonly known method. In the present invention, a feeder cell may be used, or may also be omitted from use.

The “adhesion surface” used in the present invention may also not be worked with a photoresponsive coating in which a bond between the cells and the surface is dissociated by photoirradiation. Specifically, in one aspect of the present invention, the cell adhesion surface is not given a coating using a photoresponsive material (for example, a photolytic material) which causes the cells to detach from the cell adhesion surface.

According to an embodiment of the present invention, using photoirradiation, cells can be detached from a cell adhesion surface of a culture vessel. In particular, detachment of the cells from the cell adhesion surface of the culture vessel can be performed by irradiating the cell adhesion surface with light from an interior (i.e., from the cell adhesion surface side) or an exterior (i.e., from a rear side of the cell adhesion surface) of the culture vessel.

According to an embodiment of the present invention, the cells can be detached by irradiating light from the exterior of the culture vessel without directly touching the cells themselves (i.e., in a non-contact manner). According to an embodiment of the present invention, cell detachment is possible without accessing the interior of the culture vessel, and therefore a risk of contamination to a culture system is extremely low. In addition, the photoirradiation from the exterior of the culture vessel can be performed by a mechanical operation; therefore, a process of detaching the cells from the cell adhesion surface of the culture vessel can also be automated according to an embodiment of the present invention. Moreover, according to the present embodiment, by irradiating the light selectively by region, the cells can be detached from the adhesion surface selectively by region. Cell detachment can be performed regardless of whether the culture system is an open system or a closed system because, when using the cell culture vessel in which the substrate forming the cell adhesion surface is a light-transmissive substrate, photoirradiation can be performed from outside the culture vessel. Thus, an embodiment of the present invention provides a method to detach cells from the adhesion surface selectively by region using photoirradiation in an open system culture or in a closed system culture. Moreover, in the present description, “photoirradiating selectively by region” refers to irradiating only a targeted region with light of an intensity required for cell detachment; irradiation outside of that region with light insufficient to detach cells is acceptable.

A frequency and irradiation amount of visible light irradiated so as to detach cells according to an embodiment of the present invention may be any range so long as the cells are detached from the adhesion surface. The light irradiated using a method according to an embodiment of the present invention is preferably light having a wavelength between 400 nm and 500 nm, more preferably between 400 nm and 460 nm, for example between 400 nm and 410 nm, between 410 nm and 430 nm, and/or between 430 nm and 460 nm. In a particular aspect of an embodiment of the present invention, the irradiated light has a wavelength of 405 nm, 420 nm, or 450 nm In another aspect of the present invention, the irradiated light has a wavelength between 410 nm and 430 nm, for example 420 nm. Light of this wavelength causes less damage to the cells than does UV, and has a reduced influence on cells other than those to be detached when the cells are struck by light spilling over from an irradiated region.

The cells are partially detached from the cell adhesion surface by irradiating light of 4 J or greater per 1 mm². Although the cells are not completely detached from the culture surface at a photoirradiation amount of less than 6.5 J per 1 mm², a majority do detach from the cell adhesion surface. Furthermore, at a photoirradiation amount in excess of 8.5 J per 1 mm², the cells are almost completely detached from the cell adhesion surface. Therefore, the irradiation amount of light necessary to detach the cells is preferably 4 J or greater per 1 mm², preferably 6.5 J or greater per 1 mm², more preferably in excess of 8.5 J per 1 mm², still more preferably in excess of 10.9 J per 1 mm², even more preferably 12.0 J or greater per 1 mm², and even more preferably 13.6 J or greater per 1 mm². More specifically, the irradiation amount of light necessary to detach the cells in an embodiment of the present invention can be defined as 4 J or greater, 4.1 J or greater, 4.2 J or greater, 4.3 J or greater, 4.4 J or greater, 4.5 J or greater, 4.6 J or greater, 4.7 J or greater, 4.8 J or greater, 4.9 J or greater, 5.0 J or greater, 5.1 J or greater, 5.2 J or greater, 5.3 J or greater, 5.4 J or greater, 5.5 J or greater, 5.6 J or greater, 5.7 J or greater, 5.8 J or greater, 5.9 J or greater, 6.0 J or greater, 6.1 J or greater, 6.2 J or greater, 6.3 J or greater, 6.4 J or greater, 6.5 J or greater, 6.6 J or greater, 6.7 J or greater, 6.8 J or greater, 6.9 J or greater, 7.0 J or greater, 7.1 J or greater, 7.2 J or greater, 7.3 J or greater, 7.4 J or greater, 7.5 J or greater, 7.6 J or greater, 7.7 J or greater, 7.8 J or greater, 7.9 J or greater, 8.0 J or greater, 8.1 J or greater, 8.2 J or greater, 8.3 J or greater, 8.4 J or greater, 8.5 J or greater, 8.6 J or greater, 8.7 J or greater, 8.8 J or greater, 8.9 J or greater, 9.0 J or greater, 9.1 J or greater, 9.2 J or greater, 9.3 J or greater, 9.4 J or greater, 9.5 J or greater, 9.6 J or greater, 9.7 J or greater, 9.8 J or greater, 9.9 J or greater, 10.0 J or greater, 10.1 J or greater, 10.2 J or greater, 10.3 J or greater, 10.4 J or greater, 10.5 J or greater, 10.6 J or greater, 10.7 J or greater, 10.8 J or greater, 10.9 J or greater, 11.0 J or greater, 11.1 J or greater, 11.2 J or greater, 11.3 J or greater, 11.4 J or greater, 11.5 J or greater, 11.6 J or greater, 11.7 J or greater, 11.8 J or greater, 11.9 J or greater, 12.0 J or greater, 12.1 J or greater, 12.2 J or greater, 12.3 J or greater, 12.4 J or greater, 12.5 J or greater, 12.6 J or greater, 12.7 J or greater, 12.8 J or greater, 12.9 J or greater, 13.0 J or greater, 13.1 J or greater, 13.2 J or greater, 13.3 J or greater, 13.4 J or greater, 13.5 J or greater, or 13.6 J or greater per 1 mm². In a detachment method according to an embodiment of the present invention, from a standpoint of reducing an effect on cells other than those to be detached when the cells are struck by spill-over light, the photoirradiation amount can, for example, be set to 30 J or less per 1 mm², to 25 J or less per 1 mm², to 20 J or less per 1 mm², or to 15 J or less per 1 mm². In a method according to an embodiment of the present invention, detachment of the cells after irradiation may be confirmed and further irradiation with light performed.

In a particular aspect of an embodiment of the present invention, the irradiated light is between 410 nm and 430 nm (for example, 420 nm), and the irradiation amount is 4 J or greater per 1 mm², preferably 6.5 J or greater per 1 mm², and more preferably in excess of 8.5 J per 1 mm². In this aspect of an embodiment of the present invention, the irradiation amount may be 10.9 J or greater, 12.0 J or greater, or 13.6 J or greater per 1 mm².

Output of a light source can be calculated based on the photoirradiation amount necessary for cell detachment and an amount of time required to reach the irradiation amount. The output of the light source is not particularly limited. For example, from the standpoint of administering the photoirradiation amount necessary for cell detachment in a short amount of time (for example, within 10 minutes), output at 100 mW or greater, 150 mW or greater, or 200 mW or greater is preferred.

With the objective of detaching cells specifically by region, the light irradiated to detach the cells according to an embodiment of the present invention is preferably convergent light. In an embodiment of the present invention, so long as the light is capable of irradiating the cell adhesion surface specifically by region, light converged by various methods can be irradiated. Also, an irradiated region can be defined as appropriate according to the region of the cells to be detached. In a culture of pluripotent stem cells, for example, a colony of pluripotent stem cells typically has a circular shape of between 10 μm and 2 mm in diameter. However, a need may arise to detach the cells in units of cell colonies. In order to detach the cells in units of cell colonies, the light is preferably light that is collected into a circular region of between 10 μm and 2 mm in diameter, for example, and converged to a degree enabling irradiation.

In this way, in an embodiment of the present invention, the light can be converged and irradiated to match the shape and size of the region to be irradiated. In order to converge the light, a laser light source can be used. The light irradiated from the light source may also be converged using a lens or optical fiber. The light can be converged as appropriate applying a commonly known light convergence technique.

A cell detachment method according to an embodiment of the present invention may also further include preparing a cell culture vessel in which cells are adhered to the cell adhesion surface. A cell detachment method according to an embodiment of the present invention may also include, after photoirradiation, incubating the cells. Incubation can be performed under typical cell culture conditions. An incubation time can be set to be, for example, 5 hours or longer, 10 hours or longer, 15 hours or longer, 20 hours or longer, one day or longer, or two days or longer.

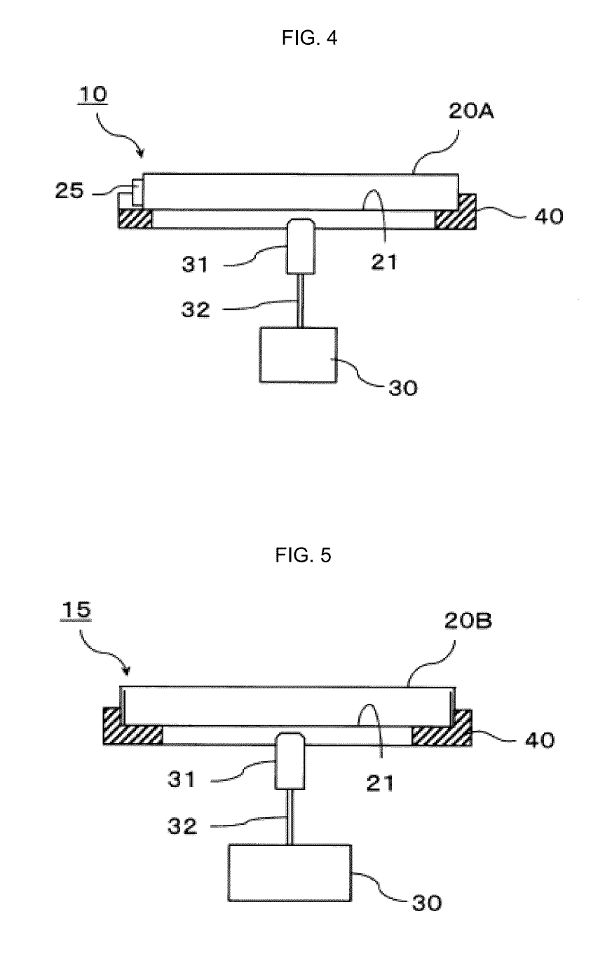

According to an embodiment of the present invention, a cell detachment system is provided that includes the cell culture vessel and the light source for cell detachment. Hereafter, with reference to FIGS. 3 and 4, a cell detachment system according to an embodiment of the present invention is described.

### First Embodiment

As shown in FIGS. 3 and 4, a cell detachment system (10) according to a first embodiment of the present invention includes a cell culture vessel (20A) having a cell adhesion surface, and a cell detachment light source (30) irradiating visible light at the cell adhesion surface. The cell culture vessel (20A) includes a vessel wall that is transparent to light irradiated from the cell detachment light source (30). The cell detachment light source (30) is provided outside the vessel wall of the cell culture vessel (20A) and can irradiate light transmitting through the vessel wall at a cell adhesion surface (21). The cell detachment system (10) according to the first embodiment includes a cell culture vessel attachment frame (40).

The cell culture vessel attachment frame (40) is a frame in which the cell culture vessel can be placed. The cell culture vessel may be either fixated, or not, to the cell culture vessel attachment frame (40). However, the cell culture vessel attachment frame (40) does not prevent the light irradiated from the cell detachment light source (30) from reaching the cell adhesion surface (21). Specifically, for example, the cell culture vessel attachment frame (40) may also be transparent to the light irradiated from the cell detachment light source (30). For example, the cell culture vessel attachment frame (40) has an area corresponding to the cell adhesion surface of the cell culture vessel, the area forming an opening so as to not prevent the light irradiated from the cell detachment light source (30) from reaching the cell adhesion surface (21) (see FIG. 4).

In the first embodiment, the cell culture vessel (20A) is a closed system culture vessel, and includes an inlet (25) to introduce the cells and culture medium into a culture chamber, and an outlet (26) to extract the cells and culture medium from the culture chamber. Alternatively, the closed system culture vessel (20A) may also include an in/outlet (27) (not shown in the drawings) combining the functions of the inlet and the outlet. The cell adhesion surface is a surface defined as noted above. The cell detachment light source (30) is provided outside the cell adhesion surface and can irradiate light at the cell adhesion surface.

The cell detachment light source (30) can, for example, be a light source irradiating light having a wavelength of between 400 and 500 nm, and can preferably be a laser light source or LED light source (for example, a laser light source or LED light source irradiating light having a wavelength of 405 nm, 420 nm, or 450 nm). The cell detachment light source (30) includes a converger (31) such as a lens to converge the light or an optical fiber (32). The output of the light source (30) is not particularly limited. For example, from the standpoint of administering a photoirradiation amount necessary for cell detachment in a short amount of time (for example, within 10 minutes), output at 100 mW or greater is preferred. In addition, the cell detachment system (10) according to the first embodiment of the present invention may also include an irradiation amount control mechanism (1A) (not shown in the drawings) controlling the irradiation amount to the cells. The irradiation amount control mechanism (1A) can stop the photoirradiation of the cells by the light source (30) when the irradiation amount reaches a predetermined value. A signal transmitter or the like can be used as the irradiation amount control mechanism (1A), for example, the signal transmitter sending a signal to the light source to automatically stop irradiating in a prescribed amount of time based on the irradiation amount from an inputter (1B) inputting the irradiation amount or irradiation time.

The cell detachment system (10) according to the first embodiment of the present invention is, in another aspect, used to detach pluripotent stem cells, and preferably to detach human ES cells or human iPS cells. Specifically, in one aspect of the present invention, the cell detachment system (10) according to the first embodiment has a pluripotent stem cell adhesion coating coated on the cell adhesion surface of the cell culture vessel (20A).

Detachment of cells using the cell detachment system according to the first embodiment of the present invention is performed with a cell detachment method according to an embodiment of the present invention.

### Modification

With reference to FIG. 5, a modification (15) of the cell detachment system according to an embodiment of the present invention is described. The modification (15) of the cell detachment system according to an embodiment of the present invention has a structure identical to that of the cell detachment system (10) according to an embodiment of the present invention, except that a cell culture vessel (20B) is an open system culture vessel or a half-open system culture vessel. In FIG. 5, identical reference numerals are assigned to portions identical to those of the cell detachment system according to the first embodiment shown in FIGS. 3 and 4, and descriptions thereof are omitted.

### EXAMPLE

### Example 1

### Detachment of Cells from Cell Adhesion Surface Using Blue Light Irradiation

In the present example, detachment of cells from a cell adhesion surface was attempted using photoirradiation.

Human iPS cells were used as the cells. A ReproFF2 culture medium was used to which 5 ng/mL bFGF (basic fibroblast growth factor) and 1% penicillin/streptomycin had been added, and was prepared under feeder-free conditions.

The culture vessels used were

a six-hole plate (Multiwell™ TC Plates-6 well, manufactured by BD & Co., product no. 353046),

a 24-hole plate (Multiwell™ TC Plates-24 well, manufactured by BD & Co., product no. 353047),

and a closed system culture vessel (OptiCell (registered trademark), manufactured by Thermo Co., product no. 155331). The cell adhesion surface of the culture vessel was coated with Matrigel (registered trademark) and used in cultivation.

A 420 nm blue LED light source (manufactured by HOYA Corporation, product no. H-1VH4-420nm) was used as the photoirradiation light source, and light was converged and photoirradiation performed at 225 mW (2.7 J/mm²). The photoirradiation of the cell adhesion surface of the culture vessel was performed from a rear side of the culture vessel bottom surface toward an interior of the culture vessel. The irradiation amount was calculated using 1 J=1 W×1 sec.

Cells were inoculated in the cell culture vessel and, after a colony formed, light having 24 intensities of between 2.7 J and 13.3 J per 1 mm² in increments of 0.5 J was respectively irradiated at the cell adhesion surface. After irradiation, the cell culture vessel was returned to a cell culturing incubator and, after 20 hours, a status of the cells was observed. A determination of whether the cells had detached was made by observing a state of cell adhesion to a bottom surface of a dish.

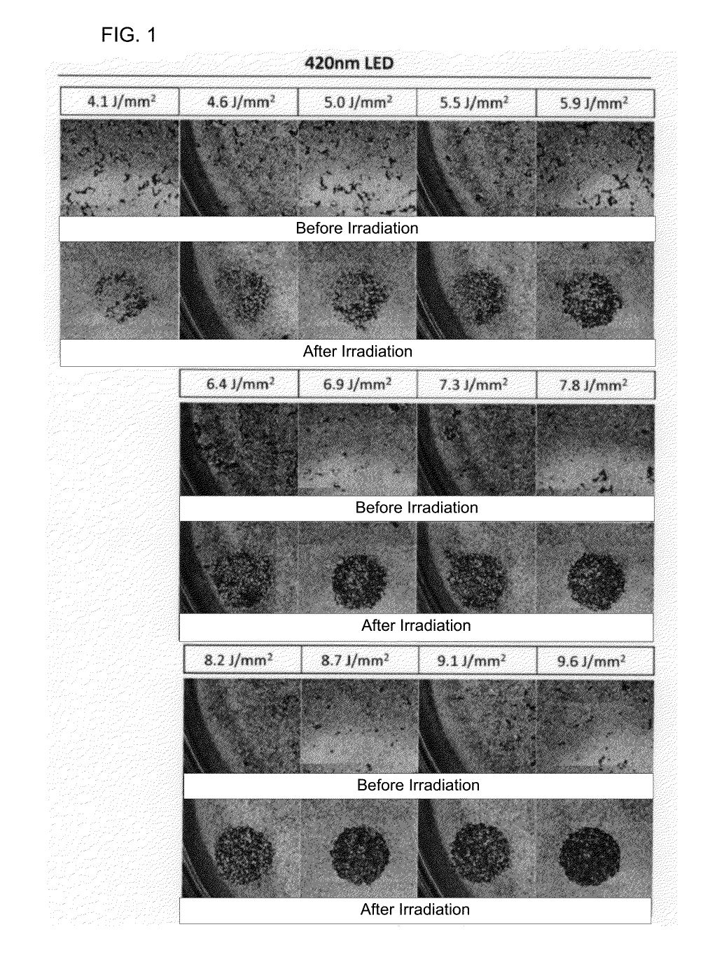

This yielded an observation that almost no cell detachment had occurred under irradiation conditions of less than 3.6 J per 1 mm², but that partial cell detachment in a photoirradiation region had occurred under the irradiation conditions of 4 J or greater per 1 mm²; favorable cell detachment had occurred under the irradiation conditions of 6.9 J or greater per 1 mm²; and almost complete detachment of cells from the cell adhesion surface had occurred under the irradiation conditions of 8.7 J or greater per 1 mm² only in those areas irradiated by the light (see FIG. 1). In addition, cells outside the photoirradiated region had not detached, and were capable of favorable cultivation thereafter (see FIG. 1). Similarly, even under irradiation conditions exceeding 9.6 J per 1 mm², almost complete detachment of cells from the cell adhesion surface had occurred only in those regions irradiated by the light (data omitted). In addition, cell detachment was observed to be promoted by the incubator. Moreover, in FIG. 1, even under irradiation conditions of 8.7 J/mm² or greater, cell detachment appears to be incomplete; however, this is due to detached, free-floating cells being captured in the photograph, and in fact complete detachment of the cells was confirmed by detailed inspection of the adhesion surface.

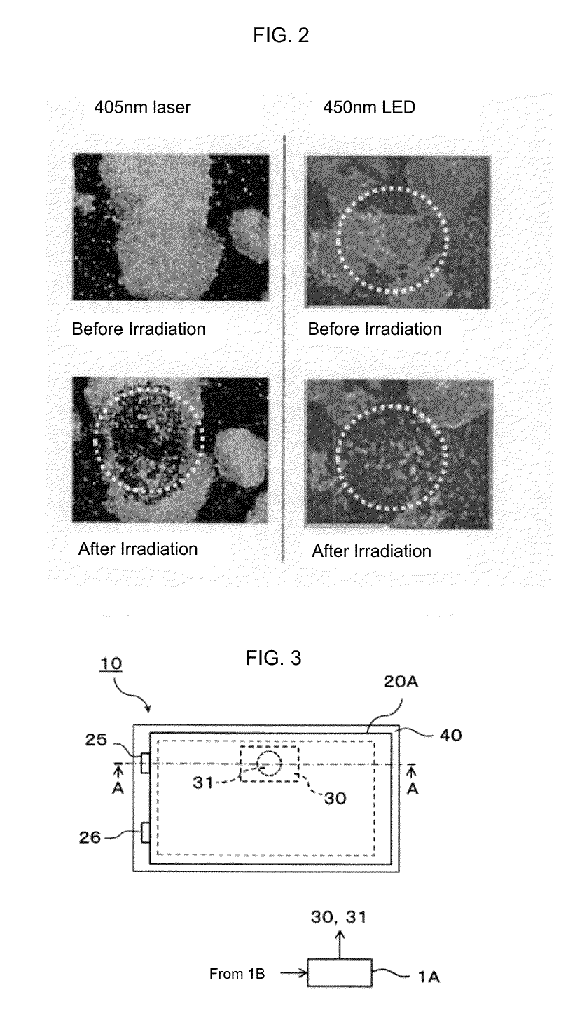

As a result, almost identical results were once again achieved even when using a light source having a wavelength of 405 nm or 450 nm (within dotted line region of FIG. 2). A laser light source (manufactured by GIGA LASER Co., product name: Violet Laser Pointer 510 V) was used as the 405 nm wavelength light source, and a blue LED light source (manufactured by HOYA Corporation, product name H-1VH4-450nm) was used as the 450 nm wavelength light source. In FIG. 2, the irradiation amount to the cells was 6.2 J/mm² at 405 nm, and 13.6 J/mm² at 450 nm.

The same result was once again achieved even when using any of the above culture vessels (data omitted). In addition, even in a case where mouse SNL cells were cultivated on a gelatin-coated culture surface, similar results were obtained (data omitted). In addition, cell detachment was promoted by the incubator (data omitted).

Accordingly, the cells were shown to be capable of detaching from the adhesion surface specifically by region using photoirradiation of 4 J or greater per 1 mm². In addition, no biological effect due to region-specific cell detachment was observed in the cells adhered to regions other than the irradiated region.

An embodiment of the present invention provides a method to detach cells in a simple manner using light.

An embodiment of the present invention provides the following.

(1) A method of detaching cells from a cell culture surface, the method including irradiating visible light having an irradiation amount of 4 J or greater per 1 mm² at the cell adhesion surface to which the cells are adhered.

(2) The detachment method according to (1), in which the irradiation amount is 6.5 J or greater per 1 mm².

(3) The detachment method according to (2), in which the irradiation amount is 8.5 J or greater per 1 mm².

(4) The method according to any of (1) to (3), in which the visible light has a wavelength of between 400 nm and 500 nm.

(5) The method according to any of (1) to (4), in which the cell adhesion surface is not coated by a photoresponsive material that causes cells to detach.

(6) The method according to any of (1) to (5), in which the light is converged and irradiated at a portion of the cell adhesion surface.

(7) The method according to any of (1) to (6), in which a vessel wall forming the cell adhesion surface is formed by a light-transmissive substrate, and in which the light is irradiated from outside the culture vessel.

(8) The method according to any of (1) to (7), in which the culture vessel is a closed system culture vessel.

(9) The method according to any of (1) to (8), in which the cells are pluripotent stem cells.

(10) A cell detachment system that includes:

a cell culture vessel having a cell adhesion surface; and

a cell detachment light source irradiating visible light at the cell detachment surface,

the cell culture vessel having a vessel wall that is transparent to light irradiated from the cell detachment light source, and

the cell detachment light source being provided outside the vessel wall of the cell culture vessel, and being capable of irradiating light transmitting through the vessel wall at the cell adhesion surface.

(11) The system according to (10), in which the light irradiated from the cell detachment light source has a wavelength of between 400 nm and 500 nm.

(12) The system according to one of (10) and (11), in which the cell adhesion surface is not coated by a photoresponsive material that causes cells to detach.

In a method according to an embodiment of the present invention, cells can be detached from the cell adhesion surface even when the cell adhesion surface is not coated by a photoresponsive material or the like.

A culture vessel having a photolytic coating on a cell adhesion surface is being developed, and a method of detaching cells selectively by region without contact using irradiation of light is being developed.

Conventionally, a cell scraper or aspirator is used to detach cells. However, in these methods, there is a risk of microbial contamination when accessing the cells. In addition, in a closed system culture vessel, physical access to the cells is difficult, and thus selective detachment of the cells is difficult.

Obviously, numerous modifications and variations of the present invention are possible in light of the above teachings. It is therefore to be understood that within the scope of the appended claims, the invention may be practiced otherwise than as specifically described herein.

What is claimed is:

1. A method for detaching cells from a cell culture surface, comprising:
irradiating visible light in an irradiation amount of 4 J or greater per 1 mm² to a cell adhesion surface to which cells are adhering on a substrate such that the cells are detached from the cell adhesion surface on the substrate.

2. The method of claim 1, wherein the irradiation amount is 6.5 J or greater per 1 mm².

3. The method of claim 2, wherein the irradiation amount is 8.5 J or greater per 1 mm².

4. The method of claim 1, wherein the visible light includes a light having a wavelength in a range of 400 nm to 500 nm.

5. The method of claim 1, wherein the cell adhesion surface does not have a coating of a photoresponsive material for detaching the cells.

6. The method of claim 1, wherein the irradiating of the visible light includes converging the visible light at a portion of the cell adhesion surface.

7. The method of claim 1, wherein the substrate has a cell culture surface on which the cell adhesion surface is formed and forms a vessel wall of a culture vessel, the substrate comprises a light transmitting substrate, and the irradiating of the visible light comprises irradiating the visible light from exterior of the culture vessel.

8. The method of claim 1, wherein the substrate has a cell culture surface on which the cell adhesion surface is formed and forms a vessel wall of a culture vessel, and the culture vessel is a closed system culture vessel.

9. The method of claim 1, wherein the cells are pluripotent stem cells.

10. The method of claim 2, wherein the visible light includes a light having a wavelength in a range of 400 nm to 500 nm.

11. The method of claim 2, wherein the cell adhesion surface does not have a coating of a photoresponsive material for detaching the cells.

12. The method of claim 2, wherein the irradiating of the visible light includes converging the visible light at a portion of the cell adhesion surface.

13. The method of claim 2, wherein the substrate has a cell culture surface on which the cell adhesion surface is formed and forms a vessel wall of a culture vessel, the substrate comprises a light transmitting substrate, and the irradiating of the visible light comprises irradiating the visible light from exterior of the culture vessel.

14. The method of claim 2, wherein the substrate has a cell culture surface on which the cell adhesion surface is formed and forms a vessel wall of a culture vessel, and the culture vessel is a closed system culture vessel.

15. The method of claim 2, wherein the cells are pluripotent stem cells.

16. A cell detachment system, comprising:
a cell culture vessel having a cell adhesion surface to which cells adhere; and
an irradiation device comprising a light source for cell detachment and configured to irradiate visible light to the cell adhesion surface on the cell culture vessel,
wherein the cell culture vessel includes a vessel wall transparent to the visible light irradiated from the irradiation device, and the light source of the irradiation device is positioned outside the cell culture vessel and configured to irradiate the visible light which transmits through the vessel wall to the cell adhesion surface of the cell culture vessel.

17. The cell detachment system of claim 10, wherein the irradiation device is configured to irradiate the visible light which includes a light having a wavelength in a range of 400 nm to 500 nm.

18. The cell detachment system of claim 10, wherein the cell adhesion surface does not have a coating of a photoresponsive material for detaching the cells.

19. The cell detachment system of claim 11, wherein the cell adhesion surface does not have a coating of a photoresponsive material for detaching the cells.

20. The cell detachment system of claim 11, further comprising:
a converging device configured to converge the visible light at a portion of the cell adhesion surface.
